(19) United States
(12) Patent Application Publication
(10) Pub. No.: US 2011/0275924 A1
(43) Pub. Date: Nov. 10, 2011

(54) Method and System for Assimilating and Transmitting Medical Imaging and Associated Data to a Remote User

(76) Inventors: William McLellan, Fort Worth, TX (US); Roberto Rodriguez Ruesga, Fate, TX (US)

(21) Appl. No.: 13/083,314

(22) Filed: Apr. 8, 2011

Related U.S. Application Data
(60) Provisional application No. 61/332,602, filed on May 7, 2010.

(51) Int. Cl. A61B 5/05

(52) U.S. Cl. 600/407

(57) ABSTRACT

The method and system of the present invention permits a physician to transmit images captured by an imaging device such as an endoscope or fluoroscope and associated data to a remote viewer in real time.

A method and system for transmitting medical imaging and associated data to a remote user begins with capturing a still or moving image (video stream) with a camera attached to a medical imaging device such as an endoscope or images generated by a fluoroscope. Such images are forwarded to and processed by a video capture device. Image related data (images data stream) is simultaneously forwarded through an in-line, one way, filter to the video capture device while the video stream is also forwarded to the video capture device.

The video and data streams are then forwarded to a data assimilating component comprising a server capable of synchronizing, compressing, and combining the streams into a compressed combined stream. This compressed combined stream is forwarded to a remote user. The remote user, upon credentialing and formatting, accesses and views the properly formatted combined data stream on a remote video display device or mobile device. Said remote user may view, simultaneously, and in real time, a plurality of image and data transmissions, such as endoscopic related information, fluoroscopic information and associated data with the use of multiple screens and/or a split screens.

This application claims the benefit of U.S. Provisional Patent Application No. 61/332,602.

### BACKGROUND OF THE INVENTION

1. Field of the Invention

The present invention relates generally to techniques for data, audio and video image communication, and more specifically to a method and system for assimilating and transmitting medical imaging and associated data to a remote user.

2. Description of the Prior Art

For a number of decades, physicians have used endoscopes to access and view interior portions of the body in order to diagnose and treat medical disorders. Such procedures, known as endoscopy, are performed using either flexible or rigid endoscopes. Endoscopes are generally comprised of a length of tubing, a light delivery system, a camera, and a channel that permits the physician to insert medical instruments.

It is not uncommon for other diagnostic studies to be taking place at the same time of an endoscopy. For example, fluoroscopic x-ray studies may be performed in conjunction with the endoscopic procedure. Therefore, multiple data sources may be available to the physician performing a procedure ranging from the endoscopic and x-ray information, to the vital signs being monitored at bed side.

During an endoscopic examination, the physician typically inserts one end of the flexible tube into either the upper gastrointestinal tract or the lower gastrointestinal tract. As the instrument is inserted, the physician is able to view images captured by the camera and projected on a display device such as a monitor while at the same time viewing other image and diagnostic information gathered by the other instruments. Occasionally, the physician will see images related to information of interest and desire to record these images on film or a hard drive for later viewing. Occasionally, the physician will ask other physicians and health care providers to view the captured images or streaming video in order to obtain treatment recommendations and advice. On still other occasions, the operating physician desires such input at the time the endoscopic or other procedure is being performed, sometimes from a physician may miles from the procedure location.

There are many devices known in the art that allow medical images and data to be transmitted to a remote location such that the images may be viewed by a remote user. Though these devices may allow medical imaging and associated data to be transmitted to remote users, they do not seamlessly allow image and data streams to be gathered, synchronized, converted to appropriate file types, so that assimilated, combined streams, are viewable, upon invitation and credentialing, by users utilizing different types of multimedia viewing platforms.

For example, U.S. Pat. No. 6,490,490 to Uchikubo teaches a remote operation support system that allows a physician in a remote location to review endoscopic images transmitted from an operating room over a communication line. A control room and operating room are linked through a public line so that an operating room operator can perform an operation while being supported by a person in the control room. The endoscope system and remote control system are linked by the public line such as an ISDN. Endoscopic signals are transmitted through the public line to a remote signal transmission apparatus.

U.S. Pat. Publication 2003/0060808 (Wilk) teaches a mobile medical facility that allows a physician to perform diagnostic and surgical operations on a patient located a distance from the physician. Images from a camera located near the patient are transmitted via a wireless antenna to a remote physician.

U.S. Pat. No. 6,699,187 to Webb describes a system that allows medical data obtained at a local site to be transferred to a data processing system located at a remote site. The data may include video data or imaging obtained from other devices such as a fluoroscope.

U.S. Pat. No. 6,791,601 to Chang describes an image capture unit that receives live video generated by an endoscopic camera and displays the video on an external monitor and stores the video within the unit. The image capture unit can also store the live video in a remote computer system via the network, as well as capturing and storing still images based on the live video and a remote computer system.

U.S. Pat. No. 7,606,861 to Killcommons teaches a medical network system that allows multimedia information to be transmitted and viewed by remote users.

U.S. Pat. Application No. 2006/0122482 (Mariotti) describes an apparatus and method to transmit, in real time, medical images over a network to a remote user.

U.S. Pat. Application No. 2007/0050828 (Renzi) describes a streaming video system that allows users to view live streaming digital video from multiple video sources in multiple operating rooms at locations connected to a network.

Although these systems and others permit medical imaging and associated data to be transmitted to remote users, there does not presently exist, a system that seamlessly allows the image and data streams to be gathered, synchronized, converted to appropriate file types, so that the assimilated, combined streams, are viewable, upon invitation and credentialing, by users utilizing different types of multimedia viewing platforms.

Therefore, it should be apparent that a need exists for an improved method and system for transmitting medical imaging and associated data to a remote user, so that image and data streams may be seamlessly gathered, synchronized, and converted to appropriate file types, such that the assimilated, combined streams, are viewable, upon invitation and credentialing, by users utilizing different types of multimedia viewing platforms.

### SUMMARY OF THE INVENTION

The present invention provides a method and system for transmitting medical imaging and associated data to a remote user. Specifically, the method and system of the present invention permits a physician to transmit images captured by an imaging device such as an endoscope or fluoroscope and associated data to a remote viewer in real time. This system allows, for example, a surgeon to view in real time, an endoscopic procedure performed by a gastroenterologist thousands of miles away.

A method and system for transmitting medical imaging and associated data to a remote user begins with capturing a still or moving image (video stream) with a camera attached to a medical imaging device such as an endoscope or images generated by a fluoroscope. Such images are forwarded to and processed by a video capture device. Image related data (images data stream) is simultaneously forwarded through an in-line, one way, filter to the video capture device while the video stream is also forwarded to the video capture device.

The video and data streams are then forwarded to a data assimilating component comprised of a server where the streams are synchronized, compressed, and combined to form a compressed combined stream. This compressed combined stream is forwarded over the internet to a remote user. The remote user, upon credentialing and formatting, accesses and views the properly formatted combined data stream on a remote video display device or mobile device. Said remote user may view, simultaneously, and in real time, a plurality of image and data transmissions, such as endoscopic related information, fluoroscopic information and associated data with the use of multiple screens and/or a split screens.

### BRIEF DESCRIPTION OF THE DRAWING

For a more complete understanding of the present invention, and the advantages thereof, reference is now made to the following descriptions taken in conjunction with the accompanying drawing, in which like numbers designate like parts, and in which:

FIG. 1 is an overall high-level block diagram of the system for assimilating and transmitting medical imaging and associated data to a remote user in accordance with the present invention.

### DESCRIPTION OF THE PREFERRED EMBODIMENT

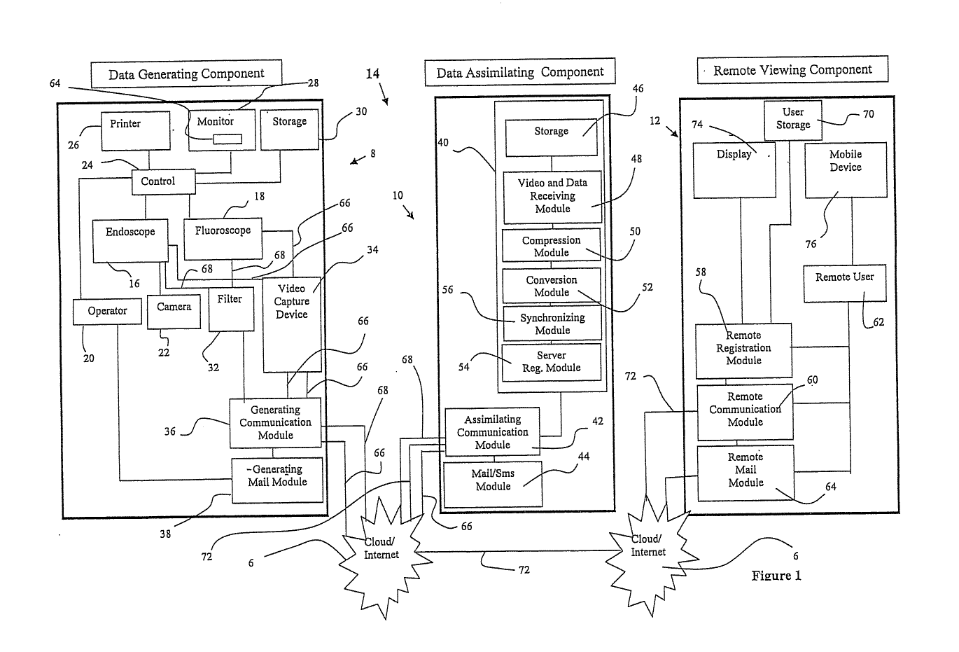

With reference now to FIG. 1, there is depicted a high-level system overview of a method and system for assimilating and transmitting medical imaging and associated data to a remote user. As illustrated, the transmission system 14 is comprised of three major components, data generating component 8, data processing component 10, and data viewing component 12.

Data generating component 8, the left side of FIG. 1, is comprised of the image and data gathering portions of the system 14. As may be seen in the FIGURE, an operator 20 such as a gastroenterologist, controls a medical imaging device 16, 18 (in a preferred embodiment, either an endoscope 16 or fluoroscope 18). In the case of the endoscope 16, the physician 20 guides the instrument by viewing an image 20 on a monitor 28, the image 64 having been generated by a camera 22 located near the end of the scope 16. Using this camera 22, the physician 20 may save moving or still images 64 of matters of interest. For example, the physician 20 may print the images 64 on a printer 26, or record the image 64 on a storage device 30.

The images 64 generated by the medical device 16, 18 are also captured by a video capture device 34. In the preferred embodiment, this video capture device is capable of receiving a video signal 68 and, if necessary, converting said signal from an analog format to a digital format. The video capture device 34 of the preferred embodiment is capable of capturing a variety of signals in a variety of different formats. For example, the video capture device 34 is capable of capturing, decoding, and deinterlacing, NTSC, PAL, MPEG 1, MPEG 2, signals as well as other standard video formats.

The data generating component 8 is further comprised of a generating communication module 36. This module 36 enables the physician 20 to forward images 64 to a corresponding communication module 42. In the preferred embodiment, images 64 are forwarded by the operator 20 to the assimilating communication module 44. The generating communication module 44 also interacts with a generating mail module 38. This module 38 is structured and arrange so as to be able to send and receive electronic messages and text messages (“messages”) through standard protocols such as Internet Message Access Protocol (IMAP), Post Office Protocol (POP), Short Message Service (SMS). In the preferred embodiment, and as will be discussed in more detail below, the operator 20 can generate a message alerting a remote user 62 to the procedure, and inviting the remote user 62 to view images 64 generated during the procedure. In addition to the images 64 generated and collected during the procedure, image related data 66 is also generated and collected. Such data 66 is comprised of information regarding the procedure and the patient. For example, such data 66 may include patient specific information such as name, age, gender, and medical condition. The data 66 may also include procedure information such as date, time, procedure name, and physician. The data 66 may also include image 64 specific information such as that which relates to the portion of the anatomy being observed, as well as the operator's 20 comments or impressions related to the procedure or the image 64 details.

Such image 64 related information 66 is forwarded along with the images 64 to the generating communication module 36. In the preferred embodiment, said image related data 66 passes through an in-line, one way, filter 32 before being processed by the generating communication module 36 to be forwarded to data assimilating component 10.

Referring to the center portion of FIG. 1, the data assimilating component 10 is comprised of server 40, assimilating communication module 42, and assimilating mail module. Server 40 is comprised of video and data receiving module 48, synchronization module 56, compression module 50, conversion module 52, server registration module 54, and storage module 46. Video and data streams 68, 66 (“communication data”) forwarded by generating communication module 36 are received via assimilating communication module 42. The generating communication module 36 and assimilating communication module 42 are comprised of standard communication ports and software applications programmed to allow communication over said ports and between said modules 36, 42 via industry standard or proprietary communication protocols. An example of such a standard communication port is a serial communication port that operates in accordance with the Electric Industry Association standard RS-232, which is an electrical and mechanical standard for a relatively short distance serial communication link. In other embodiments of the present invention, alternative communication ports may be used or extended, such as, for example, a USB port, an Ethernet port, or the like.

After receiving said communication data, the assimilating communication module forwards the streams 68, 66 to server 30. In the server 30, the streams 68, 66 are synchronized, combined, compressed, and converted to form a compressed combined stream 72. Such synchronization takes place in the synchronization module 56. This synchronization process involves taking the data 68 and video 66 streams each containing identifying data and matching the streams 68, 66 to ensure that the data portions 68 are properly matched with the video portions 66. For example, it is desirable that data portion 68 comprised of information related to a particular imaged portion of the anatomy is presented with the proper imaged portion 66. This process is analogous to synchronizing the audio and video portions of a motion picture to ensure that an actor's lips are in coordination with the sound.

Once the data 68 a video streams 66 are properly synchronized, the two streams 68, 66 are then combined to form a combined stream 72. This combined stream 72 is then compressed, using standard compression technology such as MPEG 1 and MPEG 2. Such compressed combined stream 72 facilitates storage and transmission to the remote user.

Because there are a number of different interfaces capable of downloading, streaming, and displaying video 66 and data 68 signals, the system 14 of the present invention is capable of converting the combined stream 72 to any of several common formats, depending upon the requirements of the remote user 62. For example, a remote user 62 who desires to view the combined stream 72 on a hand held device 76 might require that the stream 72 be presented in an MP4 or M4V format. The conversion module 52 is structured and arranged so as to be capable of converting the stream 72 to such formats. The conversion module 52 is capable of converting the compressed combined stream 72 to many other standard formats including, but not limited to, FLV, F4V, WMV, SWF, AVC, and the like.

This converted combined stream 72 is forwarded from the data assimilating component 10 over the internet 6 or other suitable wired or wireless conduit, to the remote viewing component 12, depicted on the right side of FIG. 1. The remote viewing component 12, is comprised of a remote communication module 60, remote mail module 58, remote registration module 58, a display device 74 which may be a mobile device 76, and a remote user 62. The remote user 62, upon credentialing and formatting, accesses and views the properly formatted combined data stream 72 on a remote video display device 74 or mobile device 76. Said remote user 62 may view, simultaneously, and in real time, a plurality of image 64, 68 and data 66 transmissions, such as endoscopic 16 related information, fluoroscopic 18 information and associated data 68 with the use of multiple screens 74, 76 and/or a split screens 74, 76.

In the preferred embodiment, the remote user 62 commands the remote registration module 58 to communicate with the server registration module 54. Remote registration module 58 and server registration module 54 include software and hardware necessary for initiating and establishing a communication link between the remote user 62 and server 40. Remote registration 58 and server registration 54 modules are further comprised of the necessary hardware and software to perform an authentication process to more securely identify the identity of the remote user 62 and the remote user 62 display capabilities. Generating communication module 36, assimilating communication module 42, and remote communication module 60 may also include hardware and software for encrypting data transmissions and messages communicated via said communication modules 36, 42, 60.

Once remote registration module 58 has transmitted to server registration module 54, the stream 72 is converted to the format compatible to the display 74, 76 capabilities of remote user 62. For example, if remote user 62 requires that the combined stream 72 be in an FLV format, remote registration module 58 communicates that information to server registration module 54 which directs conversion module 52 to convert the stream 72 to the FLV format. Such converted stream 72 is then forwarded to remote user 62 in the manner described herein.

Remote user 62 may view said converted stream 72 or store said stream 72 on user storage device 70. Similarly, all information related to the access may be stored in server storage module 46. Such information may include the raw stream 68, 66, compressed stream 72, the converted compressed stream 72 etc.

The foregoing description of a preferred embodiment of the invention has been presented for the purpose of illustration and description. It is not intended to be exhaustive or to limit the invention to the precise form disclosed. Obvious modifications or variations are possible in light of the above teachings. The embodiment was chosen and described to provide the best illustration of the principles of the invention and its practical application, and to enable one of ordinary skill in the art to utilize the invention in various embodiments and with various modifications as are suited to the particular use contemplated. All such modifications and variations are within the scope of the invention in accordance with the breadth of this disclosure, to which it is fairly, legally, and equitably entitled to be interpreted.

1. A method for assimilating and transmitting medical imaging and associated data to a remote user comprising the steps of:
capturing one or more images with a medical imaging device;
forwarding said images to a video capture device;
forwarding image related data to the video capture device;
forwarding said images and data streams to a data assimilating component, such component comprising a server capable of synchronizing, compressing and combining said images and data streams into a compressed combined stream;
forwarding said compressed combined stream to a remote user;
allowing said remote user, upon credentialing and formatting, to access and view the compressed combined data stream on a remote video display device; and
allowing said remote user to view simultaneously and in real time a plurality of image and data transmissions.

2. The method of claim 1 wherein said image related data is forwarded through a filter to the video capture device.

3. The method of claim 1 wherein said medical imaging device is an endoscope.

4. The method of claim 1 wherein said medical imaging device is a fluoroscope.

5. The method of claim 1 wherein said image related data is forwarded to said video capture device while said images are forwarded to the video capture device.

6. The method of claim 1 wherein said data assimilating component further comprises an assimilating mail module and an assimilating communication module.

7. The method of claim 6 wherein a synchronization module matches the image related data with said images to form a combined stream;
wherein said combined stream is converted to a desired format in a conversion module; and
wherein said combined stream is compressed in a compression module to form the compressed combined stream.

8. A system for assimilating and transmitting imaging and associated data, said system comprising a data generating component, a data assimilating component, and a remote viewing component, wherein:
said data generating component comprises a medical imaging device and a video capture device, wherein said video capture device is adapted to receive images generated by said medical imaging device and image related data and forward said images and image related data to the data assimilating component;
said data assimilating component comprises a server, and an assimilating communication module, wherein server is structured and arranged to be capable of synchronizing, compressing and combining said images and image related data into a compressed combined stream; and
said remote viewing component comprises a remote communication module adapted to receive said compressed combined stream and a display device capable of displaying said compressed combined stream to a remote user such that said user may simultaneously view a plurality of image and data transmissions.

9. The system of claim 8 wherein the data generating component further comprises a generating communication module capable of forwarding said images and image related data to the data assimilating component.

10. The system of claim 9 wherein the data generating component further comprises a filter through which image related data passes before being processed by said generating communication module.

11. The system of claim 8 wherein the data assimilating component further comprises a conversion module capable of converting said images and image related data to a desired format.

12. The system of claim 8 wherein the data assimilating component further comprises a server registration module and wherein the remote viewing component further comprises a remote registration module, and wherein said remote registration module is capable of communicating a desired display format to the server registration module.

13. The system of claim 12 wherein said server registration module is capable of regulating the display format to be transmitted to the remote viewing component.

14. The system of claim 8 wherein the data generating component further comprises a generating mail module capable of sending and receiving electronic messages.

15. The system of claim 8 wherein the remote viewing component further comprises a remote mail module capable of sending and receiving electronic messages.

16. The system of claim 8 wherein the data assimilating component further comprises a storage module capable of storing said images and image related data.

17. The system of claim 8 wherein said medical image device is an endoscope.

18. The system of claim 8 wherein said medical image device is a fluoroscope.

19. The system of claim 8 wherein said image related data is forwarded to said video capture device while said images are forwarded to the video capture device.
